(19) United States
(12) Patent Application Publication
(10) Pub. No.: US 2023/0218890 A1
(43) Pub. Date: Jul. 13, 2023

(54) FLEXIBLE TRANSCUTANEOUS NERVE STIMULATION DEVICE

(71) Applicant: The Procter & Gamble Company, Cincinnati, OH (US)

(72) Inventors: Erik John HASENOEHRL, Loveland, OH (US); Jeanne Marie HUGHES, Hardeeville, SC (US); Noel GAUTHIER, Cincinnati, OH (US)

(21) Appl. No.: 18/124,342

(22) Filed: Mar. 21, 2023

Related U.S. Application Data
(63) Continuation of application No. PCT/US2021/071954, filed on Oct. 21, 2021.
(60) Provisional application No. 63/108,261, filed on Oct. 30, 2020.

(51) Int. Cl. A61N 1/04

(52) U.S. Cl. CPC A61N 1/0456; A61N 1/0492

(57) ABSTRACT

The present disclosure is directed to a flexible transcutaneous nerve stimulation device for reducing urinary urgency. The devices described herein are designed to enable proper placement by a user to stimulate the user's tibial nerve, while maintaining user comfort and device functionality during wear.

### FIELD OF THE INVENTION

The present disclosure is directed to a flexible transcutaneous nerve stimulation device for reducing urinary urgency. The devices described herein are designed to enable proper placement by a user to stimulate the user's tibial nerve, while maintaining user comfort and device functionality during wear.

### BACKGROUND OF THE INVENTION

Urinary urgency is a common condition that affects many people. Urinary urgency is the sudden urge to urinate that one cannot control and can lead to wetting accidents. And, the number of people suffering from urinary urgency is likely larger than is thought, because many people living with urinary urgency do not talk to their health care provider about the condition or ask for help to treat the condition. Some are embarrassed by the condition and others do not discuss the condition or ask for help because they believe there are not any treatments for urinary urgency.

Management of urinary urgency often begins with behavioral strategies, such as fluid schedules, timed voiding, and bladder-holding techniques using a person's pelvic floor. If these behavioral strategies do not help enough, medications are available. However, many people have reservations about taking medications and medications may have unwanted side effects. Another treatment requires the surgical implantation of a nerve stimulator by a doctor or, for women, an electrical stimulation treatment may be administered via an intravaginal probe or may be worn intravaginally or perineally. These treatments can be difficult to administer or uncomfortable for the user, particularly when used for a long period of time.

Thus, there is a need for a urinary urgency treatment that is non-invasive, effective, can be applied by the user by herself or himself (without assistance from another person, including a medical professional), is less obtrusive, and is available without a prescription.

### SUMMARY OF THE INVENTION

The present disclosure relates to a transcutaneous nerve stimulation device for reducing urinary urgency in a user, the device comprising: a longitudinal centerline, a transverse centerline, at least two low motion zone-fitting regions positioned on either side of the transverse centerline, and at least one high motion zone-fitting bridge connecting the at least two low motion zone-fitting regions, wherein one or more of the at least two low motion zone-fitting regions comprises a circuitized substrate having a body-facing surface and an outward-facing surface, at least one component overlying the outward-facing surface of the circuitized substrate, at least one electrode overlying the body-facing surface of the circuitized substrate, and a power source, where the at least one component, the at least two electrodes, and the power source are electrically connected with one another for generating at least one nerve stimulating signal, the at least two low motion zone-fitting regions being electrically connected by the high motion zone-fitting bridge.

The present disclosure also relates to methods of treating urinary urgency using the devices described herein.

### BRIEF DESCRIPTION OF THE DRAWINGS

FIG. 1 is a medial view of the anatomy of a human ankle.

FIG. 2 is a medial view of a human ankle showing the dynamic and static zones of the ankle.

FIG. 3 is a top view of a transcutaneous nerve stimulation device in accordance with the disclosure.

FIG. 4 is a top view of a circuit board of a transcutaneous nerve stimulation device in accordance with the disclosure.

FIG. 5 is a bottom view of a circuit board of a transcutaneous nerve stimulation device in accordance with the disclosure.

FIG. 6. is a view of a disassembled transcutaneous nerve stimulation device in accordance with the disclosure.

FIG. 7A, 7B, 7C, 7D are exploded views of a transcutaneous nerve stimulation device in accordance with the disclosure.

FIGS. 8A and 8B are exploded views of a flexing transcutaneous nerve stimulation device in accordance with the disclosure.

FIGS. 9A-9E show transcutaneous nerve stimulation devices in accordance with the disclosure affixed to the left ankle of a user.

FIG. 10 is a block diagram of a transcutaneous nerve stimulation device in accordance with the disclosure.

FIG. 11A-11D shows the movement of the ankle and foot.

### DETAILED DESCRIPTION OF THE INVENTION

It will be understood that when an element is referred to as being “connected to” or “coupled to” or “in electrical communication with” or “electrically connected to” another element, it can be directly connected to, directly coupled to, in direct electrical communication, or directly electrically connected to the other element or intervening elements may be present. In contrast, if an element is referred to as being “directly connected” or “directly coupled” or “in direct electrical communication with” or “directly electrically connected to” another element, there are no intervening elements present.

As used herein, the term “flexible” means capable of being flexed, bent, twisted, bowed, and/or turned repeatedly, whether in-plane or out-of-plane, without breaking; pliable.

As used herein, “unitary construction” means an article of manufacture or a component thereof that is fabricated in a single piece, as opposed to fabricated by the joining together of separate pieces.

As used herein, a “body-facing surface” is a surface that, in use, faces the skin of a wearer.

As used herein, a “outward-facing surface” is a surface that faces away from the body-facing surface.

As used herein, the term “substantially free of adhesive” means that less than about 40%, less than about 30%, less than about 25%, less than about 20%, less than about 15%, less than about 10%, less than about 5%, less than about 2.5%, less than about 1%, or about 0% of the described surface area has adhesive disposed thereon.

As used herein, “integral,” when applied to portions of an article of manufacture, means that there is no joint, seam, or material boundary between the subject portions.

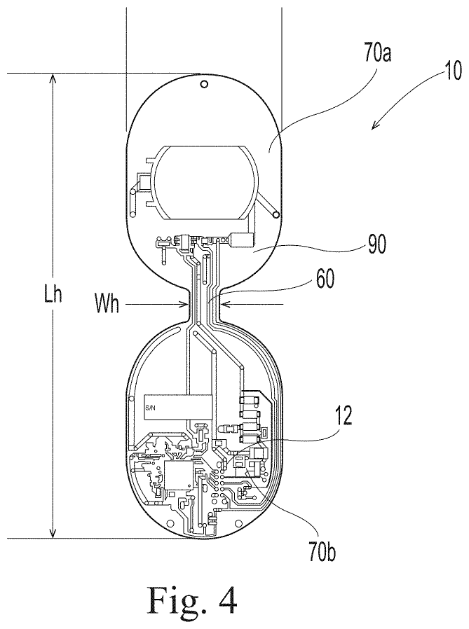

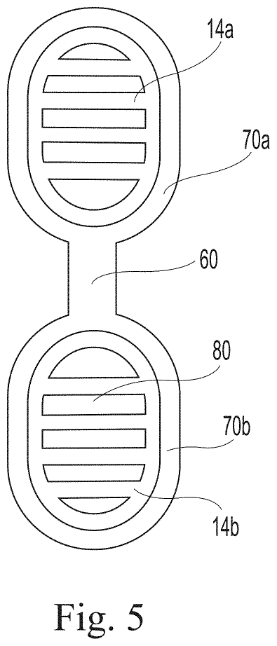

To facilitate understanding, like or identical reference numerals have been used, where possible, to designate like or identical elements common to the figures. The body-facing surface of the device and the outward-facing surface of the device are identified with reference numerals 80 and 90, respectively. For a non-unitary device, the materials or parts composing the device, such as the circuitized substrate 10, may also have a body-facing surface and an outward-facing surface. For simplicity, the body-facing surface and the outward-facing surface of the circuitized substrate 10 are identified with same reference numerals as the device, 80 and 90, respectively, in FIGS. 4 and 5, which show bottom and top views of a circuitized substrate. Also for simplicity, the body-facing surface and the outward-facing surface of every material or part composing the device is not labeled in the figures and it is understood that the body-facing surface of a part of the device faces the skin of a wearer, in use, and the outward-facing surface of a part of the device faces away from the body-facing surface.

The disclosure relates to an electrical stimulation or activation device for treatment of urinary urgency in a user, preferably a human user, via transcutaneous electrical nerve stimulation. The devices described herein are flexible, small, light, and intuitively shaped to enable proper placement by the user to stimulate the user's tibial nerve. Advantages of the devices over existing transcutaneous electrical nerve stimulation devices are: more comfortable for the user to wear, easy and intuitive for the user to apply, without the assistance of a medical professional or other person; require less power to operate; lower overall profile and a smaller footprint over a surface, such as a user's skin surface; more compact; less obtrusive; discrete; fewer parts and easier to manufacture or assemble; integrated power, communications, stimulating, and optionally sensing; inexpensive; disposable; convenient; no wires to become entangled in clothing; showerproof, sweat proof, and/or waterproof; and allows for control of stimulation parameters from a remote device, such as a smartphone or a fob, either directly by the user or by stored programs. The devices described herein effectively activate a target nerve(s), while not affecting untargeted nerves. The devices described herein are expected to have service lifetimes of days to weeks. The devices described herein may be disposable, where the disposability places less demand on power sources and battery requirements.

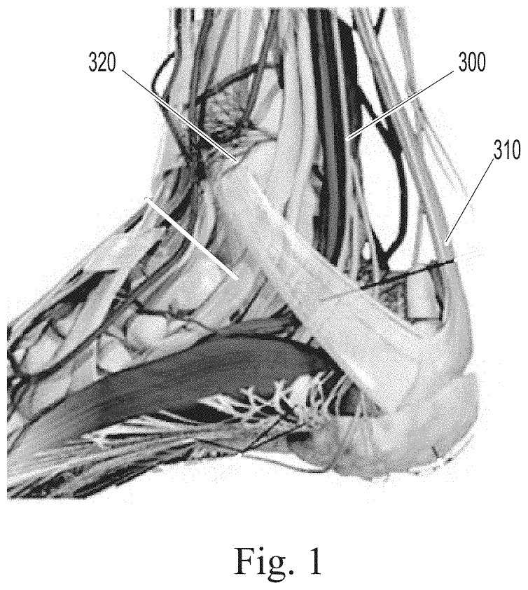

The devices described herein may be shaped and configured for placement on the medial ankle, preferably for placement proximate to the medial malleolus to stimulate the tibial nerve 300 (FIG. 1). An optimal positioning area for the electrodes of the device is believed to be on the medial or inner ankle (either ankle), proximate to and posterior to the medial malleolus 320 (FIG. 1).

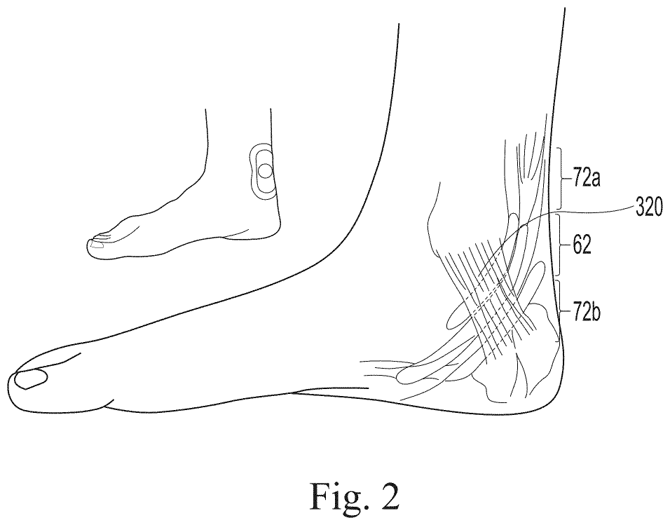

It is known that the ankle and the area surrounding the ankle (including the skin covering the ankle and the area surrounding the ankle), has dynamic or high-motion zones 62 and static or low-motion zones 72a, 72b, during movement (FIG. 2). Importantly, the optimal positioning area for the device on the ankle (proximate to and posterior to the medial malleolus 320) includes both a dynamic zone 62 and static zones 72a, 72b. As shown in FIG. 2, the medial malleolus 320 is generally a dynamic or high motion zone 62 of the ankle, while superior and inferior to the medial malleolus 320 are static or low motion zones of the ankle 72a, 72b.

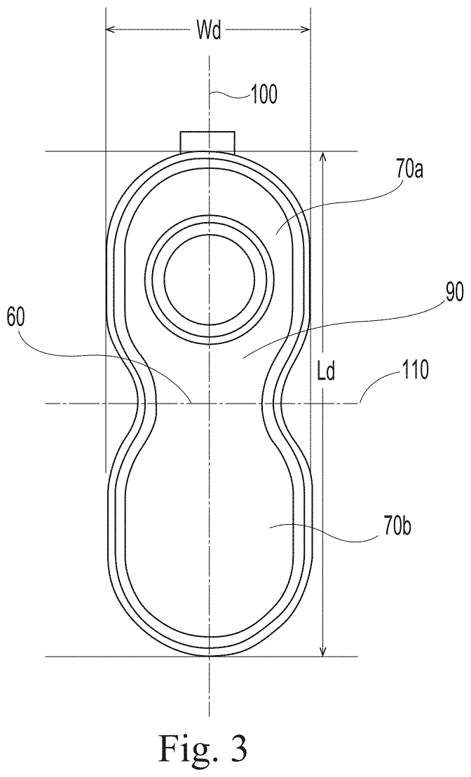

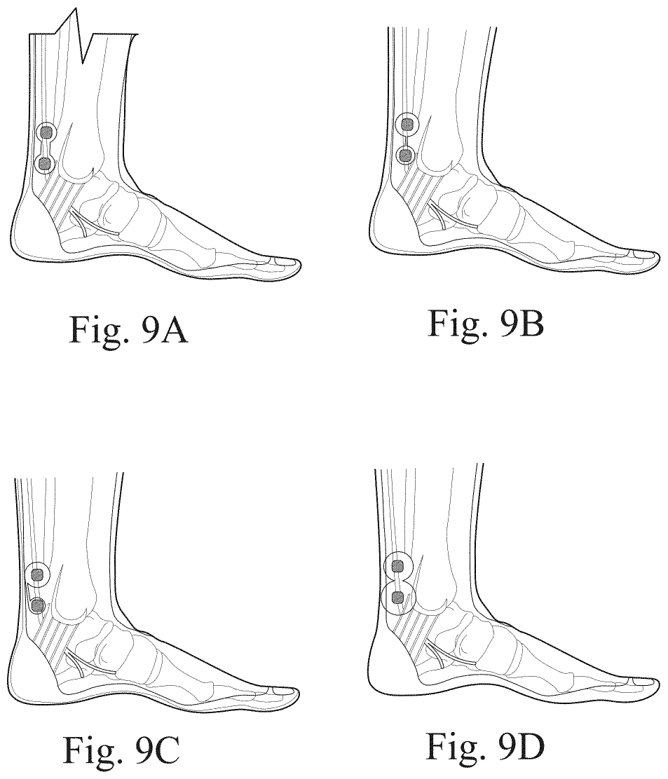

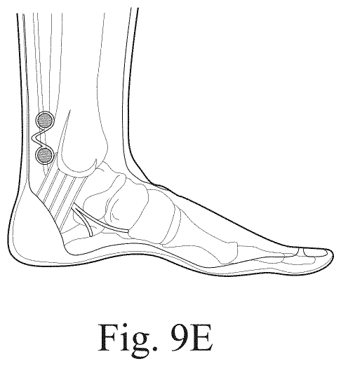

The devices described herein may comprise at least two low motion zone-fitting regions 70a, 70b and at least one high motion zone-fitting bridge 60 connecting the at least two low motion zone-fitting regions 70a, 70b, as shown in FIG. 3. In use, the at least two low motion zone-fitting regions 70a, 70b, which may comprise electrodes 14a, 14b (FIG. 5), may be affixed to static zones 72a, 72b of the ankle, for example, directly superior or inferior to the medial malleolus 320, as shown in FIGS. 9A-9E. Affixing an electrode to a static zone 72a, 72b of the ankle may provide better, more reliable contact between the electrode and the skin, which contributes to improved efficacy. The high motion zone-fitting bridge 60 may be aligned with a dynamic zone 62 of the ankle, for example, aligned with the medial malleolus 320. Aligning the high motion zone-fitting bridge 60 with a dynamic zone 62 of the ankle may provide for greater mobility of a user's ankle and, therefore, greater comfort during wear of the device. Thus, the devices described herein provide improved comfort during wear without comprising efficacy by accommodating ankle motion while maintaining reliable contact between the electrode and the skin.

The high motion zone-fitting bridge 60 may comprise an electrically conductive material that electrically connects the least two electrodes. The electrically conductive material may be a circuitized substrate 10, as shown in FIG. 4, or a wire. The high motion zone-fitting bridge 60 and each of the at least two low motion zone-fitting regions 70a, 70b may constitute separate pieces that are joined together and configured as a multi-piece element, where the high motion zone-fitting bridge 60 and each of the at least two low motion zone-fitting regions 70a, 70b are made of different materials or the same material. Alternatively, the high motion zone-fitting bridge 60 and the at least two low motion zone-fitting regions 70a, 70b may be configured as a one-piece element, for example, a one-piece, circuitized substrate 10, such as a circuit board, such as a printed circuit board.

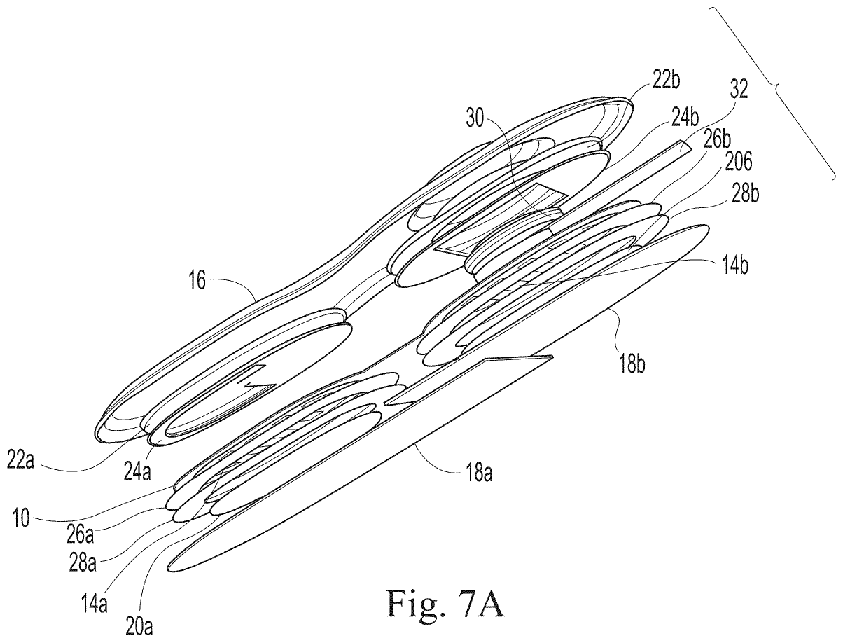

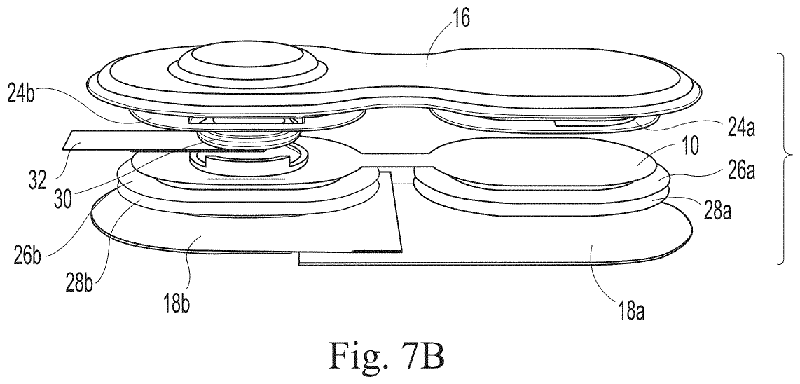

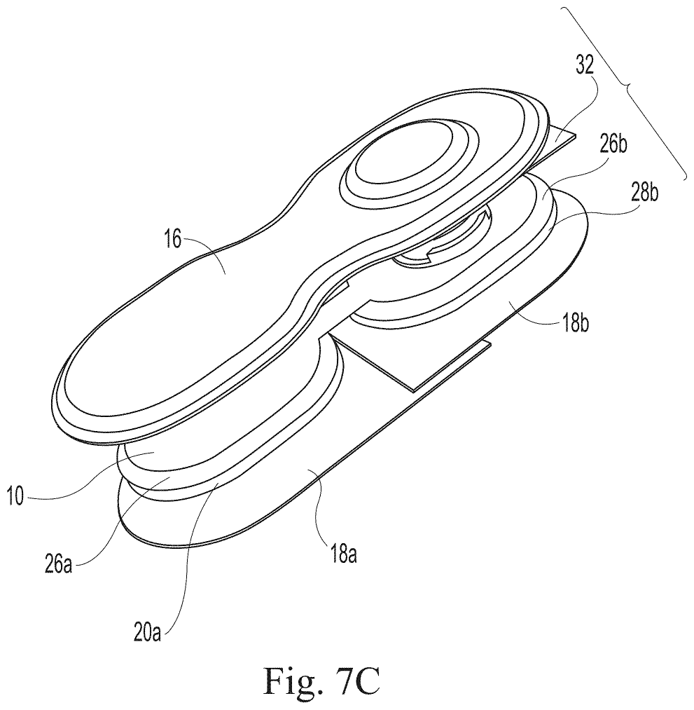

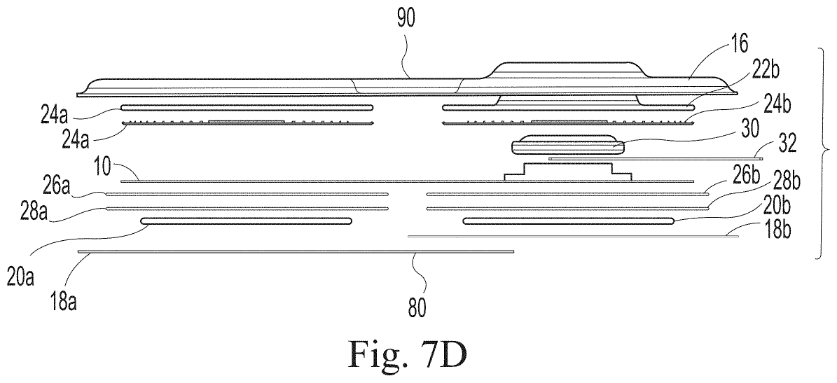

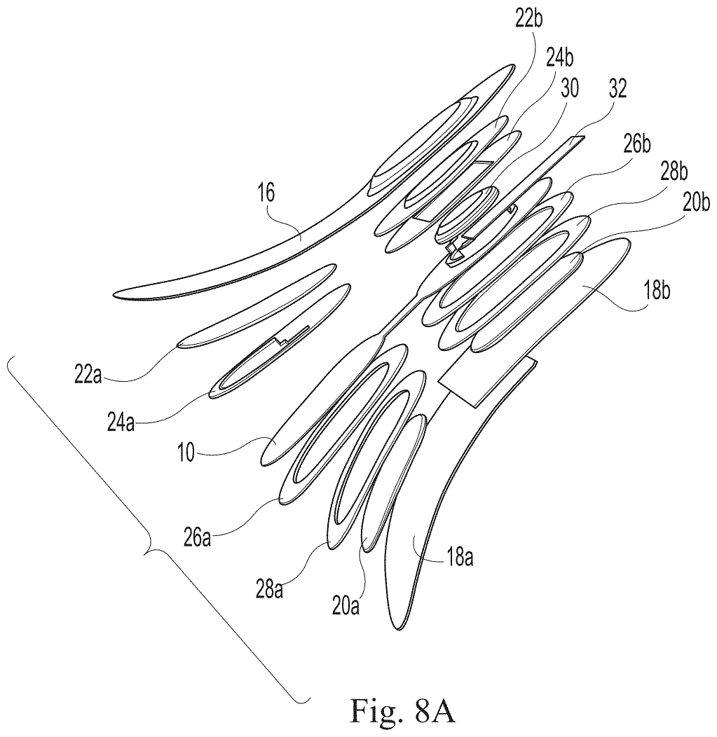

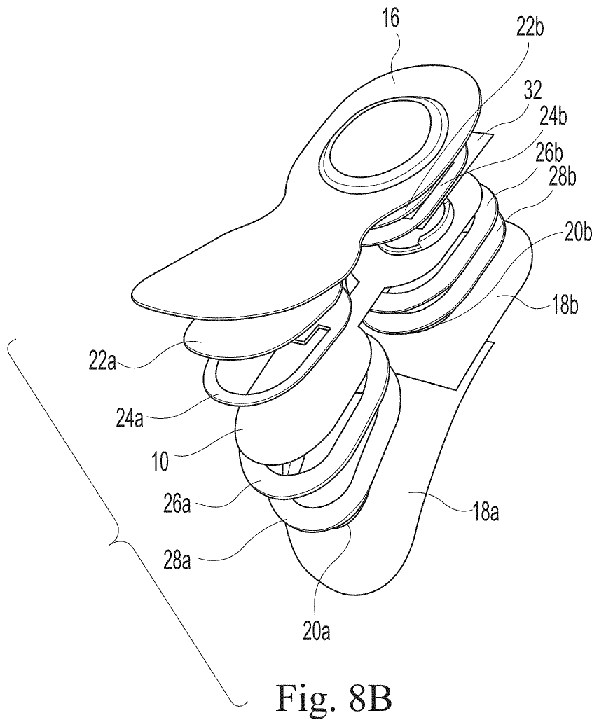

The high motion zone-fitting bridge 60 may comprise a circuitized substrate having an outward-facing surface 90 and a body-facing surface 80, where the outward-facing surface 90 and/or the body-facing surface 80 of the circuitized substrate may have an adhesive, a cushioning material, and/or a cover disposed thereon. Preferably, the high motion zone-fitting bridge 60 may comprise a circuitized substrate 10 having a cover disposed on the outward-facing surface 90 thereof. The at least two low motion zone-fitting regions 70a, 70b may each also comprise a circuitized substrate having an outward-facing surface 90 and a body-facing surface 80, where the outward-facing surface 90 and/or the body-facing surface 80 of the circuitized substrate may have an adhesive, a cushioning material, and/or a cover disposed thereon. Preferably, the at least two low motion zone-fitting regions may each comprise a circuitized substrate 10 having a cover disposed on the outward-facing surface 90 thereof. As shown in FIG. 7A, the high motion zone-fitting bridge 60 and the at least two low motion zone-fitting regions 70a, 70b are preferably configured as a one-piece circuitized substrate 10 and a one-piece cover 16 is disposed on the outward-facing surface 90 of the one-piece circuitized substrate 10, to protect the circuitized substrate and prevent the user from contacting the circuitized substrate 10. The cover 16 may have an outward-facing surface 90 and a body-facing surface 80, and an adhesive may be disposed on the body-facing surface 80 (not shown). The cover 16 disposed on the outward-facing surface 90 of the high motion zone-fitting bridge 60 may be wider than the high motion zone-fitting bridge 60, as shown in FIG. 7A, or the cover may have the same width as the high motion zone-fitting bridge 60, preferably the cover 16 is wider than the high motion zone-fitting bridge 60.

The high motion zone-fitting bridge 60 may be from about 5 mm to about 25 mm in length, preferably from about 5 mm to about 20 mm in length, more preferably from about 5 mm to about 15 mm in length. The length of the high motion zone-fitting bridge 60 may vary depending on the distance between the electrodes, which may be selected to maximize the effectiveness of the electrical signal at the target nerve and/or minimize the footprint of the nerve stimulation device. Preferably, the length of the high motion zone-fitting bridge 60 is greater than the distance between the electrodes, which may allow the high motion zone-fitting bridge 60 to accommodate ankle motion, including inversion, eversion, dorsiflexion, and plantar flexion of the ankle.

The high motion zone-fitting bridge 60 may be configured to accommodate inversion, eversion, dorsiflexion, and plantar flexion of the ankle (see FIG. 12), preferably without affecting the contact between the low motion zone-fitting regions 70a, 70b and the skin covering the static zones 72a, 72b of the ankle. The high motion zone-fitting bridge 60 may be configured to flex or bend from about 1 mm to about 20 mm, or from about 2 mm to about 15 mm, preferably from about 2 mm to about 10 mm. The high motion zone-fitting bridge 60 may be configured to flex or bend from about 1 mm to about 10 mm, preferably 2-7 mm, to accommodate dorsiflexion and/or from about 0.1 mm to 6 mm, preferably 0.1 mm to 4 mm, to accommodate eversion.

The high motion zone-fitting bridge 60 may or may not adhere to the skin covering the dynamic zone 62 of the ankle or to the skin covering the medial malleolus 320. The high motion zone-fitting bridge 60 may move independently of the skin covering the dynamic zone 62 of the ankle or the skin covering the medial malleolus 320. The high motion zone-fitting bridge 60 may lift off of the skin, float above the skin, or slide on the skin. The high motion zone-fitting bridge 60 may move independently of the low motion zone-fitting regions 70a, 70b. Preferably, the high motion zone-fitting bridge 60 does not adhere to the skin covering the dynamic zone 62 of the ankle or adheres to less than 1 mm² of skin, or less than 0.5 mm², preferably less than 0.25 mm². The body-facing surface of the high motion zone-fitting bridge 60 may or may not have adhesive disposed thereon. Preferably, the body-facing surface of the high motion zone-fitting bridge 60 is substantially free of adhesive. Less than about 40%, less than about 30%, less than about 25%, less than about 20%, less than about 15%, less than about 10%, less than about 5%, less than about 2.5%, less than about 1%, or about 0% of the body-facing surface area of the high motion zone-fitting bridge 60 may have adhesive disposed thereon. If an adhesive is disposed on the body-facing surface of high motion zone-fitting bridge 60, the peel strength of the adhesive disposed on the body-facing surface of the high motion zone-fitting bridge 60 may be less than the peel strength of an adhesive disposed on the body-facing surface of the low motion zone-fitting regions 70a, 70b.

### Nerve Stimulation Device

The transcutaneous nerve stimulation device described herein may have a unitary construction. Alternatively, the transcutaneous nerve stimulation device may have a non-unitary construction and be assembled from one or more separate parts or materials or layers thereof. For a non-unitary device, one or more of the materials composing the device may be joined together by adhesive.

With reference to FIGS. 3 and 7D, the nerve stimulation device may have two centerlines, a longitudinal centerline 100 and a transverse centerline 110, as well as a body-facing surface 80, which in use faces the skin of a wearer, and an outward-facing surface 90, which faces away from the body-facing surface. For a non-unitary device, the materials composing the device also have a body-facing surface, which in use faces the skin of a wearer, and an outward-facing surface, which faces away from the body-facing surface.

With reference to FIGS. 3, 4, and 5, the device may comprise at least two low motion zone-fitting regions 70a, 70b and at least one high motion zone-fitting bridge 60 connecting the at least two low motion zone-fitting regions 70a, 70b. The at least two low motion zone-fitting regions 70a, 70b may comprise a circuitized substrate 10 having, at least one component 12 overlying the outward-facing surface of the circuitized substrate 10 and at least two electrodes 14a, 14b overlying the body-facing surface of the circuitized substrate, the at least one component 12 and the at least two electrodes 14a, 14b being electrically connected with one another for generating at least one nerve stimulating signal.

The circuitized substrate 10 may be a printed circuit board (PCB), which may be may be rigid or flexible. A PCB having a component(s) 12 overlying a surface of the PCB may be referred to herein as a printed circuit board assembly (PCBA). As used herein, the term printed circuit board assembly (PCBA) includes the use of one or more sides of the PCB, whether rigid or flexible, for the plating of conductive paths and the mounting of electronic components.

FIGS. 7A-7D illustrate exploded views of a nerve stimulation device in accordance with the disclosure. The device may include a cover 16 having an outward-facing surface and a body-facing surface, a foam or cushion 22a, 22b, a circuitized substrate 10, at least one component 12 (shown in FIG. 4) overlying the outward-facing surface of the circuitized substrate 10, at least two electrodes 14a, 14b overlying the body-facing surface of the circuitized substrate 10, a power source 30 overlying the outward-facing surface of the circuitized substrate 10 for energizing the components, where the at least one component 12, the at least two electrodes 14a, 14b, and the power source 30 are electrically connected with one another for generating at least one nerve stimulating signal, and adhesive 24a, 24b, 26a, 26b to join two or more of the aforementioned materials to each other. Thus, the power source 30 may be disposed intermediate the outward-facing cover 16 and the circuitized substrate 10. The device may comprise a skin compatible adhesive 28a, 28b, such as various types of medical adhesive available from 3M, to affix the device to the user's skin during use. The skin compatible adhesive may or may not be electrically conductive. The skin compatible adhesive may be substantially non-electrically-conductive. Generally, if the skin-compatible adhesive is non-electrically-conductive, then high motion-fitting bridge 60 electrically isolates the at least two electrodes 14a, 14b. The skin compatible adhesive may be a hydrogel. An electrically conductive adhesive 20a, 20b, preferably a hydrogel, may be disposed on the body-facing surface of the electrode(s) 14a, 14b. The exterior cover 16 may have an adhesive disposed on its body-facing surface.

The at least one component 12 (shown in FIG. 4) overlying the outward-facing surface of the circuitized substrate 10 may be arranged in any configuration, provided that the components are electrically connected to each other. The components 12 may be arranged to maximize the flexibility of the circuitized substrate 10. The components 12 are described in more detail below. An adhesive 26a, 26b may be disposed subjacent to the circuitized substrate 10 and superjacent to the optional, skin compatible, non-electrically conductive adhesive 28a, 28b or the electrically conductive adhesive 20a, 20b disposed on the body-facing surface of the electrode(s) 14a, 14b.

FIGS. 7A-7D show adhesive 24a, 24b joining foam 22a, 22b to the circuitized substrate 10 and adhesive 26a, 26b joining the cover 16 to the to the circuitized substrate 10. Any conventional adhesive materials may be used within the device. Suitable adhesives may be made from plastic materials, nonwoven materials, silicone, acrylic, hydrogel, or latex, including polyvinylchloride, polyethylene, polyurethane. Polymeric materials suitable for use in forming nonwoven materials include polyolefins such as polyethylene and polypropylene, polyesters, nylons, ethylene vinyl acetate, ethylene methacrylate, copolymers of the above materials, and the like.

Pressure sensitive adhesives have been commonly found to work well for this purpose. The adhesive may be continuous or intermittent. For example, the adhesive may be applied in strips or across the entire surface of a particular layer. The adhesive may be applied via any suitable method, including, but not limited to spraying, printing, kiss coating, and direct slot coating.

A removable release liner 18a, 18b may be disposed subjacent to the electrically conductive adhesive 20a, 20b to protect the electrically conductive adhesive 20a, 20b, the electrode(s) 14a, 14b, and an optional non-electrically conductive adhesive, prior to use (see FIGS. 7A-7D). The release liner may be a one-piece release liner or, as shown, a two-piece release liner 18a, 18b. A two-piece release liner may allow the device to be positioned and affixed more easily by the user. In general, the thickness of the removable release liner 18a, 18b may fall within the range of about 0.05 mm to 0.20 mm. The selection of the removable release-liner 18a, 18b may depend on the type of electrically conductive adhesive used. The removable release liner 18a, 18b may comprise a polymer sheet or a paper or fabric coated with a polymer. The removable release liner 18a, 18b may have weak adhesion toward the electrically conductive adhesive 20a, 20b, thereby allowing it to be easily removed from the electrically conductive adhesive 20a, 20b prior to use without damaging the electrically conductive adhesive 20a, 20b or the electrode(s) 14a, 14b. Examples of the polymers typically used for the release liner 18a, 18b are silicones and polyethylenes. Alternatively, a wax may be used in the place of the polymer to coat the release liner 18a, 18b.

The outward-facing cover 16 may comprise a plastic material (injection molded, blow molded, thermoformed etc.), coated or uncoated paper, a nonporous film, a porous film, a woven material, a non-woven fibrous material or combinations thereof. The outward-facing cover 16 may or may not be breathable. The outward-facing cover 16 may be liquid impermeable. The outward-facing cover 16 may also be stretchable, extensible, elastically extensible, or elastomeric. The outward-facing cover 16 may be vapor permeable and yet liquid impervious. The outward-facing cover 16 may be suitable for printing.

The addition of a foam or cushion material 22a, 22b may make the device more comfortable to wear, by cushioning the power source 30 and/or the circuitized substrate 10. The cushioning material 22a, 22b may be disposed subjacent to the outward-facing cover 16 and superjacent to the circuitized substrate 10. The device may further comprise an adhesive 24a, 24b to join cushioning material 22a, 22b to the circuitized substrate 10, where the adhesive 24a, 24b may be disposed subjacent to the cushioning material 22a, 22b and superjacent to the circuitized substrate 10. Any conventional cushioning materials may be used within the device. A cushioning material may include cellulosic fibers (e.g., wood pulp fibers), other natural fibers, synthetic fibers, woven or nonwoven sheets, scrim netting or other stabilizing structures, superabsorbent material, foams, binder materials, or the like, as well as combinations thereof. Suitable cushioning materials include comminuted wood pulp, which is generally referred to as airfelt; creped cellulose wadding; absorbent gelling materials including superabsorbent polymers such as hydrogel-forming polymeric gelling agents; chemically stiffened, modified, or cross-linked cellulose fibers; meltblown polymers including co-form; synthetic fibers including crimped polyester fibers; tissue including tissue wraps and tissue laminates; capillary channel fibers; absorbent foams; absorbent sponges; synthetic staple fibers; peat moss; or any equivalent material; or combinations thereof.

The device may be designed to minimize the size and footprint of the device, e.g., lower overall profile and/or smaller footprint over the surface of the user's skin. The electrodes may be arranged in a variety of configurations. The distance between the electrodes may be selected to minimize the footprint of the nerve stimulation device (as described above).

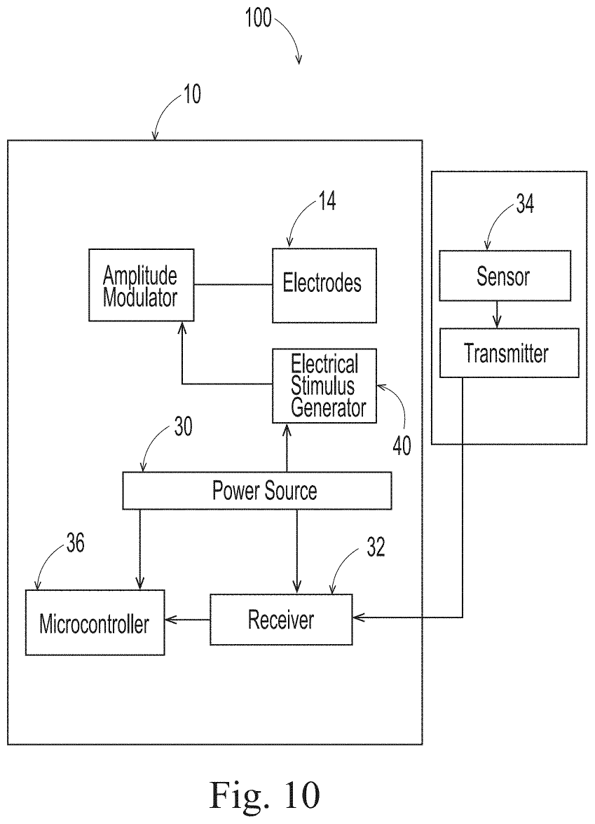

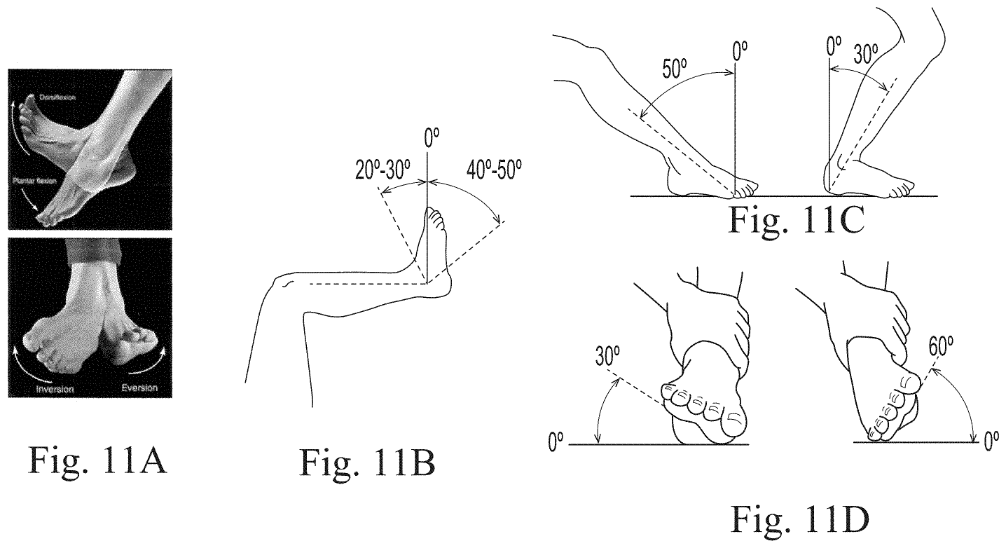

Reference is now made to FIG. 10, which is a schematic block diagram of a transcutaneous nerve stimulation device for treating urinary urgency. As seen in FIG. 10, the device 100 for treating urinary urgency may include, electrodes 14 and a power source 30 in electrical communication with the electrodes 14. When the device is activated, the electrodes 14 draw energy from the power source 30 and generate an electric field of suitable strength to stimulate the target nerve. Activation may be initiated by a user action (e.g., depressing a button on the device 100, using a remote activation device in wired or wireless communication with the device), at pre-determined times, or using biofeedback. As seen in FIG. 10, the device may include one or more components selected from the group consisting of an electrical stimulus generator 40, a signal activator 34, a microcontroller 36, a transmitter/receiver 32, an antenna (not shown), and a light emitting element (not shown).

The electrical stimulus generator 40 (also referred to as an electrical pulse generator) generates an electrical stimulus, preferably an electrical stimulus configured or adapted to stimulate a target nerve. When the electrodes 14 receive the stimulus from the generator 40, the electrodes 14 draw energy from the power source 30, e.g., battery, and generate an electrical field of suitable strength to stimulate the target nerve. The electrical field may intersect or overlap the target nerve. The electric field may stimulate the nerve by triggering its action potential, causing the nerve to send signals along its pathway. The electrical stimulus generator 40 may be of any suitable type, such as those sold by Texas Instruments of Dallas, Tex. under model number NE555.

The signal activator 34 may be in wired or wireless communication with the electrical stimulus generator 40, where the electrical stimulus generator 40 receives instructions from the signal activator 34 and where the electrical stimulus generator 40 and the signal activator 34 are in electrical communication with the power source 30. The signal activator 34 may be a single-use activator that is configured to be turned on only one time or the signal activator may be a multi-use activator. The signal activator 34 may be a switch, such as a push button switch. The signal activator may be a button on the device or the signal activator may be remote (as shown in FIG. 10), in the case of a wireless device, for example, the signal activator may be a button on a fob. A remote signal activator may have its own power source (not shown).

The device may also include an amplitude modulator 38 in electrical communication with the electrical stimulus generator, such as the modulator having the designation On-Semi MC1496, which is sold by Texas Instruments. The modulator generates a modulated waveform that is transmitted to the electrodes 14, which in turn apply the modulated waveform to the target nerve.

As seen in FIG. 10, the device 100 may include a circuitized substrate 10, a power source 30, such as a battery, and an electrical stimulus generator 40. The electrical stimulus generator 40 is electrically connected to and powered by the power source 30. The device 100 may include a control unit or microcontroller 36, which may perform functions, such as, data processing, communications, and storage. Memory may be included for storing information and instructions to be executed by the control unit (not shown). The memory can be comprised of any combination of random access memory (‘RAM’), read only memory (“ROM”), static storage, such as a magnetic or optical disk, or any other type of computer readable media. The microcontroller 36 may run software that controls the local functions of the device. The microcontroller 36 may be remote from the device, for example, the control unit may be located in a cellular telephone, a laptop, a tablet, a dedicated hardware device, such as a key fob, or some other handheld device (not shown). The device may also optionally include a transmitter/receiver, 32, for wireless external communication, and/or one or more sensors (not shown), such as, but not limited to, mechanical motion and pressure, temperature, humidity, chemical and positioning sensors. The sensor may be remote from the device, for example, a sensor may be adhered to the skin proximate to the bladder and detect the volume or pressure in the bladder. The device may also optionally include an antenna (not shown). The device may also include a light emitting element (not shown), such as an LED, for generating light signals indicating that the device is turned on.

At least one electrode is an anode and at least one electrode is a cathode, with current flowing from the anode to the cathode. Stimuli may typically be trains of voltage-regulated waves, including square waves, at frequencies between about 1 and about 150 Hz, or from about 10 to about 75 Hz, with currents between 20 and 125 mA or from about 25 to about 100 mA (at about 500 Ohms). Stimuli may be either initiated by the user when desired, or programmed according to a timed schedule, or optionally initiated in response to an event detected by a sensor that monitors some biometric of the user. The electrodes 14 may optionally collect electrical signals from the body to provide data about body functions.

Suitable electrodes 14 include both dry and floating electrodes, preferably floating electrodes. Dry electrodes are in direct contact with skin, whereas floating electrodes use electrically conductive adhesives as a chemical interface between the electrode and skin. The electrically conductive adhesive may form reliable electrical interconnections between the electrodes 14 and a user's skin and may help affix the device to the surface of a user's skin, e.g., self-adhesive hydrogel electrodes. Electrodes 14 can comprise metal, conductive polymers, electrically conductive thin films, conductive carbon, or combinations thereof. Electrically conductive adhesives are typically hydrogels. One such floating electrode is commercially available as Sticky Pad™ Surface electrode sold by Rhythmlink®. TENS Pros Premium® TENS Stim Electrodes is another example. The electrically conductive adhesive may be as thin as possible, in order to lower the profile of the device. The electrically conductive adhesive may range in thickness from about 0.5 mm to about 3.0 mm, preferably from about 0.5 mm to about 1.2 mm. The electrically conductive adhesive may be a hydrogel and the thickness of the hydrogel may be from about 0.5 mm to about 3.0 mm, preferably from about 0.5 mm to about 1.2 mm. Suitable hydrogels may have a volume resistivity of from about 200 Ohms-cm to about 1200 Ohms-cm maximum, preferably from about 340 Ohms-cm to about 1000 Ohms-cm. One such commercially available hydrogel is available as AxelGaard AG625.

Table 1 shows the number of pulses per treatment measured against two parameters, frequency and duration. Frequency is shown on the Y-axis and duration on the X-axis. Referring to Table 1, a frequency setting of 20 Hz and duration of 10 seconds produces 200 pulses.

TABLE 1

| | |
|---|---|
| Frequency | Duration (seconds) |

| (Hertz) | 1 | 5 | 10 | 20 | 30 | 60 |
|---|---|---|---|---|---|---|
| 1 | 1 | 5 | 10 | 20 | 30 | 60 |
| 5 | 5 | 25 | 50 | 100 | 150 | 300 |
| 20 | 20 | 100 | 200 | 400 | 600 | 1200 |
| 50 | 50 | 250 | 500 | 1000 | 1500 | 3000 |
| 100 | 100 | 500 | 1000 | 2000 | 3000 | 6000 |
| 150 | 150 | 750 | 1500 | 3000 | 4500 | 9000 |

The power source 30 may be a low voltage power source, providing a nominal voltage of at most 10.0 volts, at most 8.0 volts, at most 6.0 volts, at most 5.0 volts, at most 4.0 volts, or at most 3.0 volts. The power source 30 may be a battery. Batteries of different shapes and sizes may be used, where the shape and size of the battery may be selected based, at least in part, on the shape and size of the transcutaneous nerve stimulation device. Suitable batteries include alkaline batteries, silver batteries, zinc-air batteries, lithium ion batteries, lithium polymer batteries, nickel oxyhydroxide batteries, and mercury batteries. The batteries may be printable batteries, button batteries, or any other common batteries. The battery may be rechargeable or replaceable by the user. Alternatively, the battery may not be replaceable by the user (e.g., permanently mounted). The device may include a battery insulating pull tab (20) that acts as an insulator to prevent the device from activating prior to use, where the device may not be powered on until the battery insulating pull tab is removed by the user.

The battery may have a capacity of less than about 1000 milliampere hours (mAh) or from about 1 mAh to about 1000 mAh, preferably from about 1 mAh to about 500 mAh, more preferably about 1 mAh to about 100 mAh, even more preferably about 1 mAh to about 50 mAh.

Wireless Control

A user may control the nerve stimulation device using a remote wireless fob and/or other type of remote wireless device, such as a mobile phone, and a communication protocol such as Bluetooth®. The remote wireless device may include a control unit and send instructions to the nerve stimulation device and/or to a second remote wireless device (e.g., mobile phone and fob). For nerve stimulation devices having components that are remote from the device itself, such as a remote signal activator or a remote control unit, the device may require pairing with the remote component.

Using a communication protocol such as Bluetooth®, a remote signal transmitter/receiver 42 (or transceiver, e.g., Bluetooth® signal transceiver) may communicate with a nerve stimulation device that has wireless remote network connectivity. The nerve stimulation device may also communicate with the Internet or external computer networks, for example, the nerve stimulation device may transmit usage data to an external computer network for monitoring or analysis of function.

Pairing a nerve stimulation device and a remote transmitter/receiver can be instructed through a button or similar input. A control unit in the device may be instructed that an onboard signal transmitter/receiver will be paired with a transmitter/receiver in a remote device. Or, upon removal of a battery insulating tab in the device by a user, the device may enter pairing mode. The remote transmitter/receiver may enter pairing mode and may cue the user to consent to pairing. The user may consent to and initiate pairing, e.g., by depressing a button on the device. The cue to the user may include device vibration, illumination of the device, or sound.

Dimensions

The device may weigh less than about 30 g, preferably from about 1 g to about 30 g, more preferably from about 5 g to about 20 g, even more preferably from about 2 g to about 15 g. The area of the body-facing surface of the device may range from about 500 mm² to about 9000 mm², preferably about 750 mm² to about 4100 mm², more preferably about 750 mm² to about 3500 mm². The ratio of the body-facing surface area of the device to the sum of the body-facing surface areas of the electrodes may be from about 1.1:1 to about 5:1, preferably about 1.25:1 to about 4:1, more preferably about 1.5:1 to about 3:1. The device may have a maximum thickness of about 0.1 cm to about 15 cm, preferably about 0.5 cm to about 10 cm, more preferably 1 cm to about 5 cm. Thickness may be measured using any number of known methods. The device may have a maximum width (W_{d}) ranging from about 0.5 cm to about 5 cm, preferably from about 1 cm to about 3 cm, more preferably from about 1.5 cm to about 2 cm. The device may have a length (L_{d}) ranging from about 1 cm to about 10 cm, preferably from about 1 cm to about 5 cm (FIG. 3).

The high motion zone-fitting bridge 60 may have a maximum width (Wₕ) ranging from about 0.1 cm to about 0.5 cm, preferably from about 0.1 cm to about 0.4 cm. The high motion zone-fitting bridge 60 may have a length (Lₕ) ranging from about 0.1 cm to about 10 cm, or from about 0.1 cm to about 5 cm, or from about 0.1 cm to about 3 cm, or from about 0.1 cm to about 1 cm, preferably from about 0.1 cm to about 0.5 cm (FIG. 4). The high motion zone-fitting bridge 60 may have a maximum thickness (not shown) of about 0.01 cm to about 1 cm, preferably about 0.01 cm to about 0.05 cm. Thickness may be measured using any number of known methods. The area of the body-facing surface of the high motion zone-fitting bridge 60 may range from about 0.1 mm² to about 2 cm², preferably about 0.5 cm² to about 1 cm², more preferably about 0.2 cm² to about 0.7 cm².

The ratio of the maximum width (WO of the high motion zone-fitting bridge to the maximum thickness of the high motion zone-fitting bridge may be from about 100:1 to about 10:1, preferably about 90:1 to about 15:1, more preferably about 80:1 to about 20:1. The ratio of the area of the body-facing surface of the high motion zone-fitting bridge to the area of the body-facing surface of the device is from about 100:1 to about 3:1, preferably about 90:1 to about 5:1, more preferably about 80:1 to about 10:1. The ratio of the maximum width of the bridge (WO to the maximum width of the device (WO is from about 20:1 to about 1:1, preferably about 10:1 to about 2:1, more preferably about 5:1 to about 3:1.

Proper Electrode Positioning

The effectiveness of the device described herein for treating and/or managing urinary urgency may depend on proper positioning of the electrodes at the appropriate location on the user's body in order to stimulate the tibial nerve 300. Without proper positioning of the electrodes, the user may not get the full benefit or, in some cases, any benefit from the device.

The tibial nerve 300 is a branch of the sciatic nerve that passes alongside the tibia and into the foot. At the ankle, the tibial nerve 300 is relatively close to the surface of the skin. Positioning the electrodes at a location where the tibial nerve 300 is close to the skin allows for the device itself to be smaller and lighter, because less battery power is required to stimulate the nerve close to the skin. Also, there are few other (untargeted) nerves in the ankle, making it less likely that the generated electric field affects untargeted nerves.

The electrodes may be positioned on the ankle in a variety of ways, however, not all positions are equally effective or efficient. For example, it is not preferred to position one or more of the electrodes on the lateral side of the ankle, as the tibial nerve 300 is not close to the skin on the lateral side of the ankle. Also, positioning on the lateral side of the ankle may activate pain nerves. Positioning the electrodes above the ankle is not preferred, as the tibial nerve 300 is covered by muscle above the ankle, which requires more energy is required to activate the nerve. Positioning the electrodes on the top or bottom of the foot is not preferred, as the tibial nerve 300 branches out in the foot, making it more difficult to activate at this location (also such positioning may cause discomfort while walking or standing). Also, the electrodes may be positioned transversely across the path of the tibial nerve 300 or axially along the path of the tibial nerve 300. If the electrodes are arranged transversely across the nerve, then more battery power may be required to stimulate the nerve. The electrodes are preferably arranged axially along the nerve (as shown in FIGS. 9A-9E), such that the electrical field generated by the electrodes intersects or overlaps the tibial nerve 300. An optimal positioning area for the electrodes is believed to be on the medial or inner ankle (either ankle), proximate to and posterior to the medial malleolus 320.

Communicating to the user where to affix the device, such that the electrodes are positioned in the desired locations on the ankle, may be challenging. Instructions including writing, illustrations, or orientation marks for properly affixing the device may be included on the device itself, in the packaging for the device, or on the packaging of the device. However, users may dispose of the instructions without reading them, for example, when disposing of the packaging, or simply ignore the instructions. An effective way to communicate to a user where to affix the device, such that the electrodes are positioned in the desired location on the ankle, may include selecting one or more anatomical landmarks on the ankle and making the shape of the device complement the anatomical landmark(s). Selecting an anatomical landmark(s) on the ankle and designing the shape of the device to complement or complete the landmark, may make the device more intuitive for the user to affix. Suitable anatomical landmarks include the achilles tendon 310, the heel 330, the arch 340, the medial malleolus 320, the lateral malleolus (not shown), or combinations thereof, as seen in FIG. 1. The arch 340 and/or the medial malleolus 320 are preferred. The medial malleolus 320 is most preferred. These anatomical landmarks on the ankle are prominent and fairly easy for the user to identify, by sight or touch.

Shape

The sides and edges of the ankle are generally nonlinear. As used in the context of the present specification, the term “nonlinear” refers to any of various curved, as opposed to straight, lines. As such, the devices described herein may have at least one nonlinear edge, which may align or substantially align with a nonlinear edge of one of the anatomical landmarks described herein, preferably the medial malleolus 320. The medial malleolus 320 is a circular protrusion on the medial side of the ankle. As used in the context of the present specification, a circle has one edge—its circumference. As shown in FIGS. 9A-9E, a nonlinear edge of the device may be concave relative to the medial malleolus 320 and posterior to the medial malleolus 320. That is to say, a nonlinear edge may curve around the posterior border (or posterior edge) of the medial malleolus 320.

### Assembly

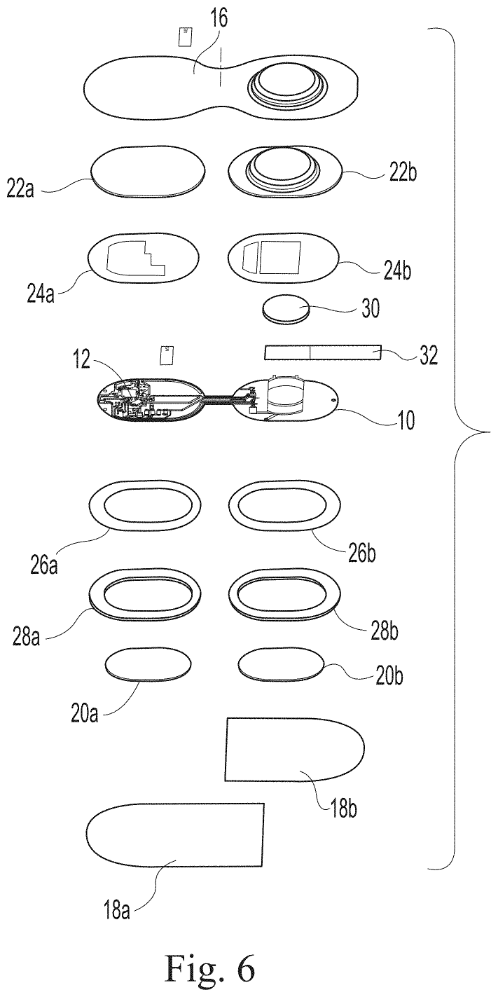

For a non-unitary device, the materials composing the device may be assembled using conventional techniques known in the art for constructing and configuring assembled articles. FIG. 6 shows a disassembled transcutaneous nerve stimulation device in accordance with the disclosure.

### Camouflaging

The transcutaneous nerve activation device described herein may be camouflaged to make the device less obtrusive and more discrete. Camouflaging includes matching the color of the device to the skin tone of the user, matching the color of the device to a user's garment, or disguising the device as something less upsetting to the user, such as a blister pad. Camouflaging may also include a very colorful, bright device or a device having a colorful design, to make the device look like an accessory or a tattoo.

### Method of Treating Urinary Urgency

A method for treating urinary urgency may comprise the steps of: providing a transcutaneous nerve stimulation device in accordance with the disclosure; affixing the device to the medial ankle of a user, preferably a user experiencing urinary urgency; activating the device to stimulate the tibial nerve of the user.

### Instructions for Use

Instructions including writing, illustrations, or orientation marks for properly aligning and affixing the device may be included on the device itself, in the packaging for the device, or on the packaging of the device. With regard to proper positioning of the device, an effective way to communicate to a user to properly affix the device, such that the electrodes are positioned in the desired location on the ankle, is by selecting one or more anatomical landmarks on the ankle and making the shape of the device complement or complete the anatomical landmark(s). By selecting an appropriate anatomical landmark(s) on the ankle and then by designing the shape of the device to complement or complete the landmark, a device that is intuitive for the user to affix properly is provided. Suitable anatomical landmarks include the achilles tendon 310, the heel 330, the arch 340, the medial malleolus 320, the lateral malleolus (not shown), or combinations thereof, as seen in FIG. 1. The arch 340 and/or the medial malleolus 320 are preferred. The medial malleolus 320 is most preferred. These anatomical landmarks on the ankle are prominent and fairly easy for the user to identify, by sight or touch.

Numerous suitable nerve stimulation devices, including components of the devices and protocols for operating the devices, are described in U.S. patent application Ser. No. 15/912,058, and U.S. Pat. No. 10,016,600, both of which are hereby incorporated by reference. A nerve stimulation device that is shaped and configured to be readily, properly placed by a user on his or her ankle is described in US Patent Application No. 20190117974.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A transcutaneous nerve stimulation device for reducing urinary urgency in a user, the device comprising: a longitudinal centerline, a transverse centerline, at least two low motion zone-fitting regions positioned on either side of the transverse centerline, and at least one high motion zone-fitting bridge connecting the at least two low motion zone-fitting regions, wherein one or more of the at least two low motion zone-fitting regions comprises a circuitized substrate having a body-facing surface and an outward-facing surface, at least one component overlying the outward-facing surface of the circuitized substrate, at least one electrode overlying the body-facing surface of the circuitized substrate, and a power source, where the at least one component, the at least two electrodes, and the power source are electrically connected with one another for generating at least one nerve stimulating signal, the at least two low motion zone-fitting regions being electrically connected by the high motion zone-fitting bridge.

2. The transcutaneous nerve stimulation device of claim 1, wherein the ratio of the maximum width of the at least one high motion zone-fitting bridge to the maximum thickness of the at least one high motion zone-fitting bridge is from about 100:1 to about 10:1.

3. The transcutaneous nerve stimulation device of claim 1, wherein the ratio of the area of the body-facing surface of the at least one high motion zone-fitting bridge to the area of the body-facing surface of the device is from about 1:100 to about 1:3.

4. The transcutaneous nerve stimulation device of claim 1, wherein the ratio of the maximum width of the at least one high motion zone-fitting bridge to the maximum width of the device is from about 1:1 to about 1:20.

5. The transcutaneous nerve stimulation device of claim 1, wherein the device is shaped and configured for placement on the medial ankle.

6. The transcutaneous nerve stimulation device of claim 1, wherein the at least one high motion zone-fitting bridge is configured to accommodate dorsiflexion and plantar flexion of the ankle.

7. The transcutaneous nerve stimulation device of claim 1, wherein the at least one high motion zone-fitting bridge is configured to accommodate inversion and eversion of the ankle.

8. The transcutaneous nerve stimulation device of claim 1, wherein the body-facing surface of the at least one high motion zone-fitting bridge is substantially free of adhesive.

9. The transcutaneous nerve stimulation device of claim 1, wherein an adhesive is disposed on the body-facing surface of device and the peel strength of the adhesive disposed on the body-facing surface of the at least one high motion zone-fitting bridge is less than the peel strength of the adhesive disposed on the body-facing surfaces of the at least two low motion zone-fitting regions.

10. The transcutaneous nerve stimulation device of claim 1, wherein an adhesive that is compatible with skin is disposed on the body-facing surface of the device.

11. The transcutaneous nerve stimulation device of claim 1, wherein the device is disposable.

12. The transcutaneous nerve stimulation device of claim 1, wherein the device does not travel the circumference of the ankle.

13. The transcutaneous nerve stimulation device of claim 1, wherein the at least one component is selected from the group consisting of an electrical stimulus generator, a microcontroller, an amplitude modulator, memory, a signal activator, and a transmitter/receiver.

14. The transcutaneous nerve stimulation device of claim 1, wherein the at least two electrodes are positioned along the longitudinal centerline of the device.

15. The transcutaneous nerve stimulation device of claim 1, wherein none of the electrodes are configured to be positioned on the top of the foot, on the sole of the foot, or on the lateral ankle.

16. The transcutaneous nerve stimulation device of claim 1, wherein the device comprises at least one nonlinear edge.

17. The transcutaneous nerve stimulation device of claim 1, wherein the body-facing surface area of the device is from about 500 mm² to about 9000 mm².

18. The transcutaneous nerve stimulation device of claim 1, wherein the maximum thickness of the device is about 0.10 mm to about 15 mm.

19. The transcutaneous nerve stimulation device of claim 1, wherein the device weighs from about 1 g to about 30 g.

20. A method for treating urinary urgency comprising the steps of:
(a) providing a transcutaneous nerve stimulation device according to claim 1;
(b) affixing the device to the medial ankle of a user;
(c) activating the device to stimulate the tibial nerve of the user.
